(19) United States
(12) Patent Application Publication
(10) Pub. No.: US 2016/0199361 A1
(43) Pub. Date: Jul. 14, 2016

(54) Method of Inducing An Anti-Retroviral Immune Response By Counter-Acting Retro-Virus Induced Anti-Apoptosis

(71) Applicant: RUTGERS, THE STATE UNIVERSITY OF NEW JERSEY, New Brunswick, NJ (US)

(72) Inventors: Hartmut M. Hanauske-Abel, Edgewater, NJ (US); Paul Palumbo, Westfield, NJ (US); Bernadette M. Cracchiolo, Edgewater, NJ (US); Axel R. Hanauske, Hamburg (DE); Michael B. Mathews, Montclair, NJ (US)

(21) Appl. No.: 14/913,169

(22) PCT Filed: Aug. 19, 2014
(86) PCT No.: PCT/US14/51737

§ 371 Date: Feb. 19, 2016

Related U.S. Application Data
(60) Provisional application No. 61/867,542, filed on Aug. 19, 2013.

(51) Int. Cl. A61K 31/4412; A61K 31/4418

(52) U.S. Cl. CPC A61K 31/4412; A61K 31/4418

(57) ABSTRACT

A method of inducing an anti-retroviral immune response by counter-acting retro-virus induced anti-apoptosis is disclosed. The present invention relates, at least in part, to agents and methods for treating, inhibiting, vaccinating or controlling HIV. In certain non-limiting aspects, it relates to the reduction of viral load in an HIV-1 infected subject, while simultaneously developing immunological responsiveness within the subject toward HIV-1 that continues after the agent is removed or excreted from the subject's body.

### CROSS REFERENCES TO RELATED APPLICATIONS

This application claims priority of U.S. Provisional Application No. 61/867,542, filed on Aug. 19, 2013, the contents of which are incorporated herein by reference in its entirety.

### GOVERNMENT INTERESTS

The invention disclosed herein was made, at least in part, with Government support under Grant Nos. HD-1457, AI034552 and AI060403 from the National Institutes of Health. Accordingly, the U.S. Government has certain rights in this invention.

### FIELD OF THE INVENTION

The present invention relates, at least in part, to reducing or terminating of HIV infection in a subject while simultaneously vaccinating the subject against the virus.

### BACKGROUND OF THE INVENTION

The immune system of humans and of animals is optimized to defend the integrity of the human and the animal body against exogenous and endogenous pathogens. If unchecked, these pathogens will disrupt the physiological structure and function of said bodies, resulting in illness and death of an affected individual and often causing the most severe consequences for public health and the economy. The pathogens inactivated by the various parts of an individual's immune system, which comprises and integrates innate and adaptive responses, include but are not limited to: (i) microbial agents like viruses, bacteria, fungi, parasites, and their products; (ii) tissue-damaging molecules (‘toxins’) of variable origin; and (iii) cells of the human and the animal body that have suffered alterations of the genes controlling proliferation and differentiation so that, in the form of premalignant and malignant lesions (cancer), these cells become a threat to the survival of an affected individual.

In the course of human history, pathogens not inactivated by the various parts of an individual's immune system have caused catastrophic events of extraordinary proportions. Three examples are cited as evidence for the human and societal costs of immune defense failure at the level of individuals.

Influenza: The 1918 H1N1 pandemic (‘Spanish flu’), which infected one third of the world population, caused the death of at least 50 million, and likely 100 million, individuals. This loss of human life is comparable to the estimated combined casualties of the First and the Second World War.

Smallpox: During just the past century, smallpox reportedly caused the death of half a billion individuals. This loss of human life probably exceeds the total casualties of all wars on Earth between 1900 and 2000.

Rinderpest: A highly contagious disease of hoofed animals, especially cattle, mortality can approach 100% in immunologically naïve herds, with severe economic impact on human populations.

The world-wide threat of Spanish flu, smallpox, rinderpest, and of at least 30 other infections, has been overcome by vaccination: Current anti-flu vaccines protect against the 1918 strain; anti-smallpox vaccines used in the Smallpox Eradication Program of the World Health Organization terminated the natural occurrence of causative Variola variants in 1979, just like the Global Rinderpest Eradication Program of the Food and Agriculture Organization of the United Nations eradicated the rinderpest morbillivirus in 2011.

The term ‘vaccination’ designates the administration to an individual of exogenous material (‘vaccine’) derived from an exogenous or endogenous pathogen. Such administration is performed with the therapeutic intention of activating the innate and adaptive responses of that individual's immune system for clinical effect, the latter consisting in the quantifiable reduction or elimination of the pathogen in the body of an exposed individual, with resultant amelioration or prevention of morbidity and mortality caused by said pathogen. The relative contributions of the various parts of a vaccine-activated immune system to pathogen reduction, and thus the actual immunological mechanism(s) involved, may vary between pathogens and individuals, but irrespective of molecules, pathways, and cells involved, the one constant and consistent outcome is the reduction of the very pathogen that without vaccination would escape from the response of the immune system.

Vaccinations are generally performed with protein or nucleic acid molecules isolated from, and representing strategic elements of, a pathogen of interest. This approach is rendered ineffective if the pathogen is able to undergo escape mutations and rapidly generates pathogenic yet immunologically distinct and non-crossreactive progeny. Such situations are encountered with several viruses, in particular HIV-1.

Viruses, upon infection of a host cell, invariably suppress its genetically preprogrammed response of self-destruction (‘apoptosis’), in this way denying its synthetic machinery for use by the invading virus to generate infectious progeny. The anti-apoptotic activity of viruses forces infected cells to stay alive and keep on functioning for the purpose of virion production and spread of infection. This activity also blinds the immune system of an infected individual since apoptotic cells are known to be, under certain conditions when produced ex vivo in the manner of a vaccine, effective enhancers of immunogenicity of the viral molecules they contain.

Modeled on the numerous favorable precedents for the public health impact of vaccinations, significant efforts were made during the past decades to develop a vaccine that protects against infection with human immunodeficiency virus 1 (HIV-1). Such a vaccine is supposed to induce the autonomous, endogenous suppression of the viral load of HIV-1, persistent long after administration and precisely in the same manner any vaccine is known to do by clinical precedent. From 2000 to 2012, this effort received $9.342 billion in support and will break the $10 billion mark by 2013. This funding generated more than 50 vaccine candidates that progressed to human trials, yet only in very few cases did the results justify further progression to large-scale testing. The results of the latter were marginal at best and usually failures:

The VAX004 trial relied on recombinant envelope protein (rgp120) as modeled by the successful strategy against hepatitis B. However, that strategy had shown no efficacy in a similar, rpg120-employing large-scale test (VAX003). The trial confirmed the induction of marked immune responses, but also their absent effect on viral load and their failure to prevent HIV-1 acquisition. A subpopulation of vaccine recipients was found to display HIV-1 acquisition significantly higher than in the placebo controls (relative risk 1.78, P=0.026). This outcome was termed an “undeniable fiasco”.

The STEP trial had to be halted for nonefficiency at the first interim analysis despite being highly effective for inducing cellular immune reactivity against HIV-1. Instead of reducing viral load, and thus protecting against HIV, a large subset of vaccine recipients suffered an enhanced risk of HIV acquisition relative to the placebo controls.

The RV 144 trial received huge publicity, claiming to establish the precedent that vaccination against HIV-1 is possible, and identified per intent-to-treat analysis “a trend toward the prevention of HIV-1 infection among vaccine recipients, with a vaccine efficacy of 26.4%”. The statistical procedures were immediately called into question and subsequently found to be consistent with “≧22% chance remaining for no efficacy.”

The HVTN 505 trial, begun in 2009, was discontinued in April 2013 for failing to reduce viral load and to prevent HIV infection in vaccine recipients. Instead of reducing viral load, and thus protecting against HIV, a subset of vaccine recipients appeared to suffer an enhanced risk of HIV acquisition relative to the placebo controls.

Remarkably, the clusters of viral targets recognized by the immune system after HIV-1 vaccination (‘immunodominant epitope hotspots’) uniformly differ from those elicited by natural infection with HIV-1. Even more, in all trials analyzed—Merck16, STEP, and HTVN 054—the administered vaccine induced immune reactivity to epitope hotspots that located, preferentially or exclusively, to genetically highly variable and thus infection-irrelevant sites of viral products. By contrast, natural infection with endogenous HIV-1 induced immune reactivity mostly to epitope hotspots that are genetically invariable and thus by evolutionary evidence indispensible for the infectivity of the virus. In summary, immunization by endogenous HIV-1 antigens generates an immune response that demonstrably differs from the one induced by vaccination with exogenous HIV-1 antigens, the latter being insufficiently effective or ineffective at HIV-1 suppression (no viral load reduction), as reported for VAX003, VAX004, STEP, RV144, and HVTN 505; or even advantageous to HIV-1 acquisition (preferential infection of vaccine recipients), as observed in VAX004, STEP, and HVTN 505.

The vaccine failures in trials like VAX003, VAX004, STEP, RV144, and HVTN 505 made it impossible to address the basic issue of whether the genetic diversity of HIV-1 can be overcome by any vaccination protocol: a vaccination can deliver only a fixed number of exogenous antigens and thus is based on the premise that the endogenous infection-relevant antigens are similar or identical to those in the vaccine. However, this premise does not hold for HIV-1: The reverse transcriptase of HIV lacks proofreading activity, the ability to confirm that the DNA transcript it makes is an accurate copy of the RNA code, and confers a mutation rate of approximately 3.4×10⁵ mutations per base pair per replication cycle. Since the HIV genome is an estimated 10⁴ base pairs in length and the baseline rate of viral production is approximately 10¹⁰ virions per day, millions of viral variants are produced within any infected person in a single day. HIV-1 recombination can lead to further viral diversity and occurs when one person is coinfected with two separate strains of the virus that are multiplying in the same cell. The genetic diversity due to high mutation, high recombination, and high replication of HIV-1, and thus the variability of this virus' nucleotide sequence is recognized in the art as a major determinant for the specificity and sensitivity of diagnostic tests; for the acquisition and transmittal of resistance to antiretrovirals; and for antigenicity and immunogenicity of apparent ‘consensus’ sequences. Furthermore, despite displaying the same mutation rate in different human cells, HIV-1 is known to generate cell type-specific mutation spectra, resulting in the emergence of not just patient-specific pseudo-strains of HIV-1, but within a patient in organ-specific genotypes.

In light of the seemingly inexhaustible genetic diversity of HIV-1, which generates patient-specific and even cell type-specific variants, and the experimental evidence for an inherent inability of exogenous HIV-1 antigens to target all infection-relevant structures of the virus, the utility of vaccine development and administration in order to curtail HIV-1/AIDS faces principle and unique limitations, as established by the consistently reproduced vaccine failures in large-scale testing. While there are certainly drug-based methods of reducing viral load, such as those disclosed in published PCT International Application No. WO 1995/27485, no vaccine has been produced, or is disclosed in this reference, to achieve a reduction in viral load or a clinically relevant protection against HIV acquisition. Antiretroviral drugs and their combinations (‘combined antiretroviral therapy’, or cART) do achieve these effects as long as they are properly applied: in uninfected individuals, cessation of their administration voids pre-exposure prophylaxis (PrEP)-based protection against infection; and in infected individuals, cessation of their administration causes rapid rebound of the viral load to pre-treatment levels, generally within a few days of drug discontinuation.

In view of these facts there remains an urgent need for HIV-1/AIDS treatments, particularly treatments with long-lasting effects.

### SUMMARY OF THE INVENTION

The present invention relates, at least in part, to agents and methods for treating, inhibiting, vaccinating or controlling HIV. In certain non-limiting aspects, it relates to the reduction of viral load in an HIV-1 infected subject, while simultaneously developing immunological responsiveness within the subject toward HIV-1 that continues after the agent is removed or excreted from the subject's body.

In certain non-limiting aspects, the invention relates to a method of inducing an anti-retroviral immune response by counter-acting retro-virus induced anti-apoptosis. Such method includes administering a therapeutic agent selected from deferiprone, ciclopirox, hydralazine and combinations thereof to a subject infected with HIV-1 in an effective amount and for a time period effective to allow infected cells to present HIV-1 antigens for immunological stimulation, followed by discontinuing administration of said deferiprone, ciclopirox or hydralazine after said effective time period, whereby viral load decreases during the administration and continues to decrease after the deferiprone, ciclopirox or hydralazine is excreted from the subject's body. Deferiprone, ciclopirox or hydralazine is administered, in certain aspects, such that it is provided at a concentration in the subject's serum of at least about 150 μM. In further aspects, it is provided at a concentration of at least about 200 μM. Such concentrations may be maintained in the subject for any length of time consistent with the teachings herein. In certain aspects, it is substantially maintained (i.e. within about 1%, or about 5%, or about 10%, or about 25% of the desired concentration) for at least one week. In further aspects, it is substantially maintained until a decline in expression of the human genome-integrated HIV DNA, as monitored by p24 or HIV RNA levels, or a reduction in that viral DNA itself is detected in a living patient receiving such drug by any route that causes and maintains the required systemic levels, e.g. by the oral or intravenous route.

Deferiprone, ciclopirox or hydralazine may be administered in any dosage, particularly any dosage that obtains the desired concentration level in the subject. In certain non-limiting aspects, the dosage administered is from about 30 mg per kg bodyweight to about 150 mg per kg bodyweight, distributed over 24 hours in such manner as to provide a concentration in the subject's serum of at least about 150 μM.

In further embodiments, the present invention includes a method of inducing an anti-viral immune response by limiting self-tolerance protection of viruses. Such method includes administering a therapeutic agent selected from deferiprone, ciclopirox, hydralazine and combinations thereof to a subject infected with HIV-1 in an effective amount and for a time period effective to allow infected cells to present HIV-1 antigens for immunological stimulation, followed by discontinuing administration of said deferiprone, ciclopirox or hydralazine after said effective time period, whereby viral load decreases during the administration and continues to decrease after the deferiprone, ciclopirox or hydralazine is excreted from the subject's body. The dosaging, concentration provided to the subject, timeline of administration may be consistent with the teachings herein, such as those provided above.

In even further embodiments, the invention includes a method of inducing an anti-retro-viral immune response by counter-acting retro-virus induced anti-apoptosis by administering a therapeutic agent selected from deferiprone, ciclopirox or hydralazine to a subject infected with HIV-1 in an amount and for a time effective to (i) activate apoptosis preferentially in HIV-infected cells; (ii) inhibit HIV-1 gene expression and therefore, provide temporary relief from its immunosuppressive products; and (iii) limit the HIV-1 protecting self-tolerance via suppression of Clq biosynthesis; and discontinuing administration of said deferiprone, ciclopirox or hydralazine with resultant continuation of viral suppression.

Additional modifications, embodiments, and advantages will be readily apparent on the basis of the remaining disclosure.

### BRIEF DESCRIPTION OF THE DRAWINGS

FIG. 1 displays the dose-dependent antiretroviral kinetics of deferiprone in isolate-infected, long-term replenished primary cell cultures. Virological parameters in long-term replenished primary cell cultures treated with 200 μM deferiprone for the indicated periods, with post-treatment observation periods of 10 days (A) and 87 days (B). The data suggests that the viral RNA decline may continue off treatment at the on-treatment rate.

FIG. 2 displays the deferiprone disruption of self-stabilized HIV-1 production in isolate-infected, long-term replenished primary cell cultures. Dose-dependent suppression of HIV-1 protein and HIV-1 RNA generation during 35 days of treatment with deferiprone. At 100 μM deferiprone, viral RNA rebounds rapidly above the level attained upon cessation of drug, increasing at a rate of log₁₀ +0.24/ml/day up to the set-point level in co-incubated HIV-infected untreated control cultures (green line segments). At 200 μM deferiprone, viral RNA rebound is absent off drug. Up to five days post drug cessation, the decline of viral RNA appears to continue at the on-drug rate of log₁₀ −0.04/ml/day, and viral RNA does not rebound above the level attained at cessation of drug.

FIG. 3 shows acute HIV-1 suppression by deferiprone in vivo. In HIV-1 infected treatment-naïve persons, an acutely suppressive effect on HIV-1 RNA occurs coincident with intake of the medicine if a threshold concentration in serum is attained. The left-sided graphic for groups A and B depicts deferiprone drug levels obtained with individuals achieving ≧150 μM in Group A and <150 μM in Group B. The right-sided graphic depicts viral levels at baseline and after even days of treatment.

FIG. 4 shows persistent HIV-1 suppression after deferiprone cessation in vivo. In HIV-1 infected treatment-naïve persons, the acutely suppressive effect on HIV-1 RNA in responsive subjects persists at four and seven weeks after treatment discontinuation. Individuals are grouped by whether or not their viral levels had decreased at Day 7 of treatment. Longer term monitoring off treatment ensued. The double-arrowed symbols, color-coded for each individual in the ‘Response’ cohort, highlight the HIV-1 RNA level relative to that individual's pre-treatment value.

FIG. 5 shows apoptosis of PBMCs acutely infected with HIV-1 upon treatment with 30 micromolar ciclopirox.

### DETAILED DESCRIPTION OF THE INVENTION

Infection of an individual by HIV-1 is known to involve several mechanisms that combine to render the individual's immune response ineffective against the virus, with the virus then proceeding to dismember and destroy the immune system.

As noted above, despite extraordinary effort all attempts to develop a protective vaccine against HIV-1 have failed so far, and immunological methods to fight the virus are not forthcoming. However, it is known that the use of HIV-infected apoptotic cells for vaccination, instead of purified protein or nucleic acid antigens of HIV-1, generates protective immunity against challenge with infectious doses of HIV-1 in at least one accepted animal model for the human disease. See, for example, U.S. application Ser. No. 13/271,190, the contents of which are incorporated herein by reference in its entirety.

The pharmacological methods of fighting the virus are much more advanced and constitute the only medical approach of pragmatic relevance. Yet even this approach is significantly limited by several factors, in particular the emergence of strains resistant to many or all clinically employed antiretrovirals and the inability to eradicate the virus once it has infected an individual.

Surprisingly, is has now been discovered that the low molecular weight oral metal chelating drug deferiprone (3-hydroxy-1,2-dimethylpyridin-4(1H)-one, and referred to herein as “DEF”) inhibits the rebound of the HIV-1 viral load for months after ingestion had been discontinued and the compound excreted. This inhibition can also be obtained with ciclopirox, which is also a low molecular weight metal chelating drugs, and hydralazine, which is a smooth muscle relaxant vasodilator. This long-lasting suppression of HIV-1 is dose-dependent; occurs only in patients who in their plasma achieve a critical drug concentration threshold that causes a decrease of their viral load while on drug; and said decrease persists off-drug despite complete drug elimination from the human body, as evidenced by the fact that said persistent suppression lasted at least up to 200 times the half-life of said medicine in the human body. Consequently, its total elimination rules out any direct involvement of said medicine in the post-cessation continuous suppression of HIV-1 viral load. The measurements indicate that deferiprone treatment induces an autonomous endogenous suppression of the viral load of HIV-1, said suppression being persistent long after cessation of administration, precisely in the same manner any vaccine is known to act by clinical precedent. Since no administration of any vaccine occurred and the prolonged suppressive off-drug effect is identical to that of a vaccination by triggering the emergence of autonomous endogenous suppression, this drug-based modality is herein termed ‘vaccineless vaccination’.

Deferiprone, ciclopirox and hydralazine are representative example of compounds that causes vaccineless vaccination in HIV-1 infected patients. As long as deferiprone, ciclopirox or hydralazine is present at the threshold concentration of 150 μM, the drugs show the ability to inhibit the expression of the HIV genome and to cause the ablation of HIV-infected cells by apoptosis. However, it is completely unexpected that the compounds would provide a suppressive activity on the viral load of HIV-1 when it is no longer administered, and after clearance from the body.

Thus, in one embodiment of the invention, drugs that counteract the viral anti-apoptosis and thus release infected cells from the virally imposed block, such as deferiprone, ciclopirox or hydralazine, are used for the in situ unmasking of a wide array of viral epitopes in such apoptotic cells and consequently, for the in vivo generation of an immune response that at least limits infectivity for the viral quasispecies that evolved in a particular patient. This is particularly applicable to the treatment of HIV-1/AIDS. A further embodiment of the invention is directed to treatment of viral infections in general.

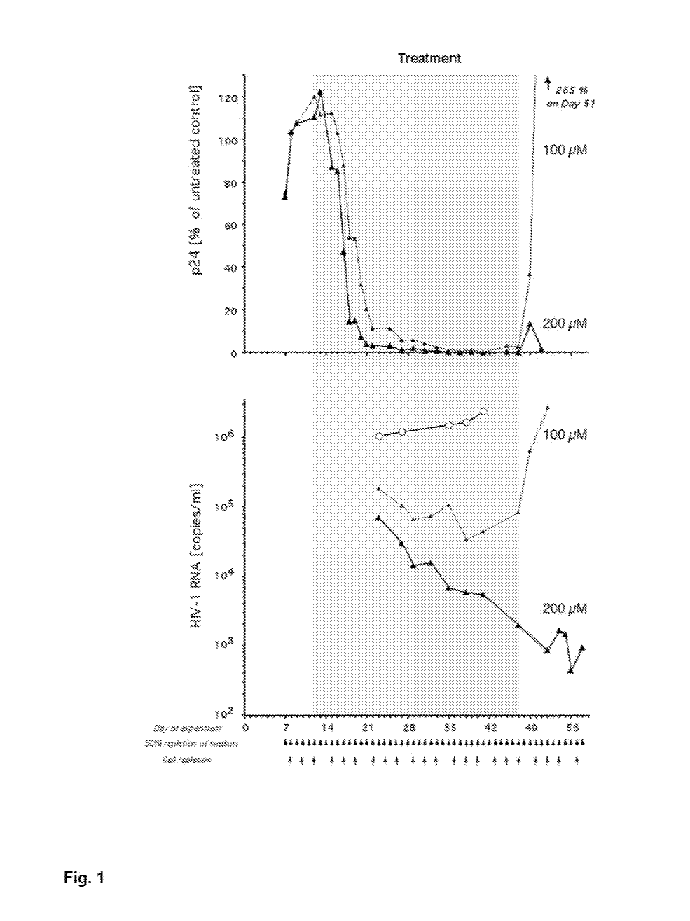

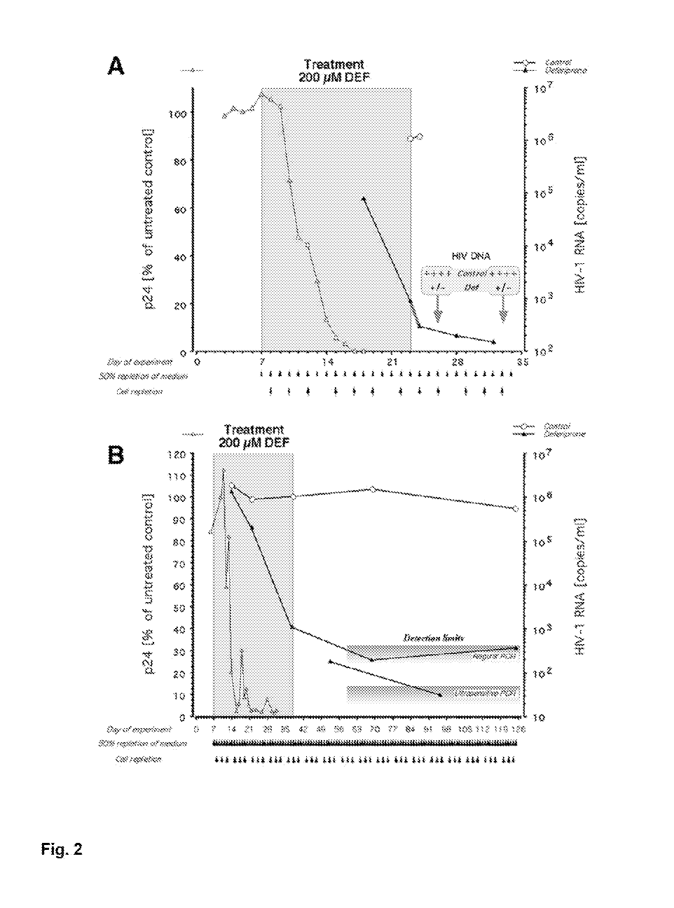

The existence of an antiretrovirally effective threshold concentration between 100 μM and 200 μM (FIG. 1) and, upon drug cessation, the lack of rebound to the pre-treatment set-point of viral RNA copies (FIG. 2A,B) informed the design of a double-blind placebo-controlled pilot human trial. An in vivo threshold at ≧150 μM for antiretroviral activity was hypothesized since in infected T-cell lines, effective suppression of HIV-1 generation and selective induction of apoptosis both require at least 150 μM deferiprone, ciclopirox or hydralazine. The minimal antiretroviral activity of deferiprone, ciclopirox or hydralazine monotherapy for a week should, only in individuals with serum concentrations above this threshold, cause a viral load decline at least in the range of zidovudine monotherapy, i.e. −0.3 log₁₀, an apparently minor change that nevertheless reduces the annual risk of progression to AIDS-related death by 25%.

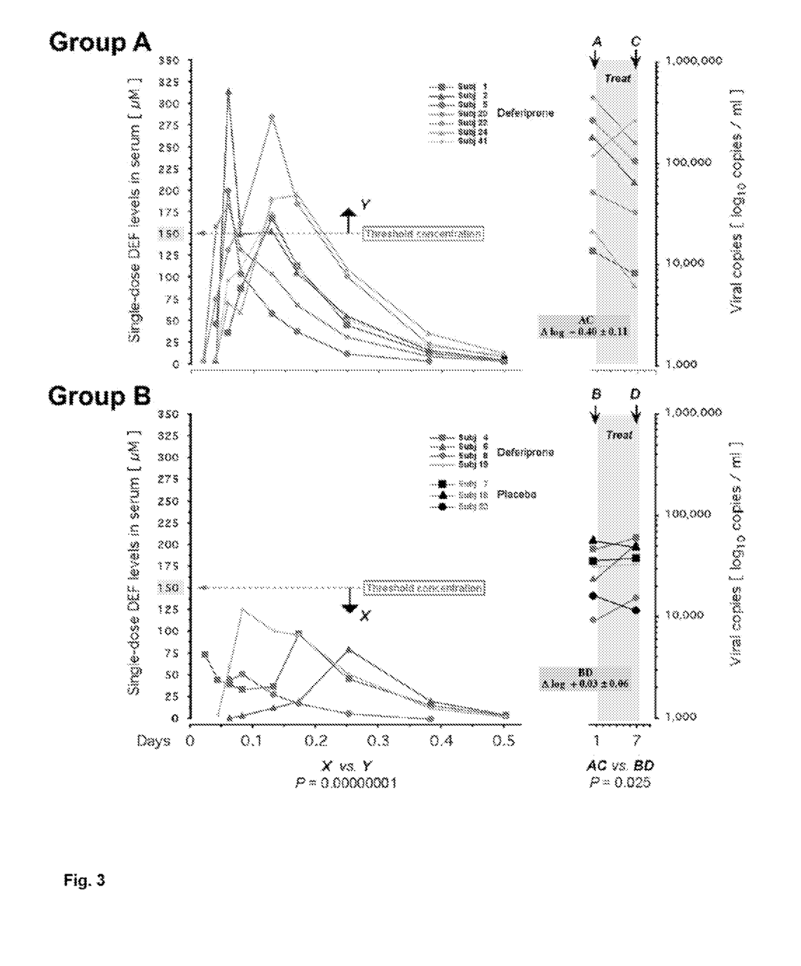

In the IRB-approved pilot trial, HIV-1-infected but treatment-naïve individuals who achieved ≧150 μM in serum (Group A) on either 33 mg/kg po TID or 50 mg/kg po TID oral deferiprone (N=7), demonstrated a modest virological effect during the seven-day [D] treatment [Δ log₁₀ D7 vs. D1: −0.40±0.1], and 6 of 7 experienced an acute viral load decline [Δ log 10 D7 vs. D1: −0.42±0.05]. By contrast, the cohort that did not achieve ≧150 μM in serum (Group B), receiving either placebo (N=3) or deferiprone (N=4), did not show an acute virological effect [Δ log 10 D7 vs. D1: −0.07±0.05 and +0.17±0.07, respectively] (FIG. 3). Individuals in the 50 mg/kg po TID group did not tolerate the regimen, resulting in protocol disruptions and early terminations, yet invariably only deferiprone peak concentrations ≧150 μM coincided with an acute decline in HIV-1 RNA (Δ log 10=−0.44±0.1). The viral response distribution relative to the serum deferiprone levels among the 11 treated subjects is significant (P=0.0152) by two-sided Fisher's Exact test and thus, consistent with the hypothesis of an antiretrovirally effective in vivo threshold at ≧150 μM deferiprone.

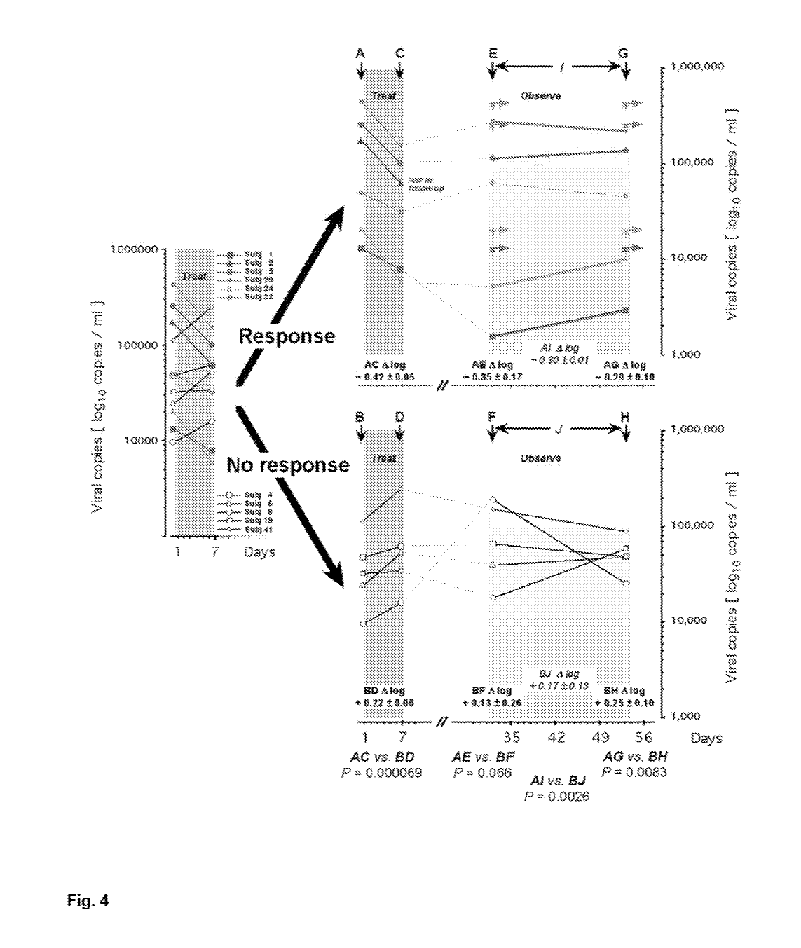

To analyze the pilot trial in a hypothesis-independent manner, the two-stage discontinuation trial design (DTD) was followed. In a first stage of predetermined duration, all subjects receive the new agent; in the second stage, subjects are segregated into ‘responders’ and ‘non-responders’ and their subsequent outcomes are followed separately in order to avoid dilution of the primary data for treatment benefit, if it exists. This enrichment approach is descriptive and ignorant of mechanistic knowledge. The results of the DTD-based analysis for deferiprone are shown in FIG. 4. At the end of the first stage, the 6 responders and the 5 non-responders differed by Δ log 10=0.64 (D7 vs. D1, P=0.00007). At the end of the second stage, the 5 responders—one was lost (Subject 2)—and the 5 non-responders differed by Δ log=0.54 (D53 vs. D1, P=0.008) even though one responder (Subject 22) had reacquired the pre-treatment level.

In summary, 80% of acute responders to the brief, one-week deferiprone pulse did not reacquire their viral load baseline for seven weeks post-treatment, i.e. at 200 times the half-life of said medicine in the human body and thus assuredly after its total excretion, indicating the acquisition of autonomous endogenous suppression of the HIV-1 viral load, and thereby producing “vaccineless vaccination”. The acquisition of autonomous endogenous suppression, as in any vaccination, is further evidenced by the fact that the viral load is not just blocked from rebounding, but can decline further, entirely off drug and “on its own”, as evidence by subjects #1 and 24 (FIG. 4).

While not intending to be bound by theory, it is believed that apoptotic cells cover themselves with a molecule that cloaks them from being recognized as immunogens by the immune system. This molecule, Clq, is avidly bound by the gp41 element of HIV-1 as well as by human cells undergoing apoptosis. Cells undergoing apoptosis are a most common event in the human body and, as long as these dying cells are covered by Clq, they do not elicit immune system activation against self-antigens. However, hereditary defects of Clq expression in humans lead invariably to autoimmune disease; in fact, such deficiencies make human Clq one of the gene products conveying the strongest disease susceptibility for autoimmune diseases in humans. With Clq cloaking being in effect, the explanation presented above would therefore be invalid.

It is believed that deferiprone, ciclopirox and hydralazine inhibit the key event in Clq biosynthesis, the posttranslational hydroxylation of specific prolyl residues in its collagen-like domains by the enzyme prolyl hydroxylase. Of note, deferiprone-treated thalassemic patients display signs of immune activation and launch immune responses against self-epitopes. The mechanistic concept for vaccineless vaccination against HIV-1 therefore comprises at least three elements: (i) activation of apoptosis preferentially in HIV-infected cells; (ii) inhibition of HIV-1 gene expression and therefore, temporary relief from its immunosuppressive products; and (iii) limitation of the HIV-1 protecting self-tolerance via suppression of Clq biosynthesis.

Based, at least in part, on the foregoing, the present invention includes a composition that contains a suitable carrier and at least the compound deferiprone, ciclopirox or hydralazine, which may be administered to a subject infected with HIV-1 in an effective amount or dosage to reduce the viral load and develop an innate immunological response within the subject to HIV-1. The composition can be a pharmaceutical composition that contains a pharmaceutically acceptable carrier. The term “pharmaceutical composition” refers to the combination of an active agent with a carrier, inert or active, making the composition especially suitable for diagnostic or therapeutic use in vivo or ex vivo. The term “pharmaceutically acceptable carrier” refers to any of the standard pharmaceutical carriers, such as, but not limited to, a phosphate buffered saline solution, water, emulsions, and various types of wetting agents.

The compositions also can include stabilizers and preservatives. A pharmaceutically acceptable carrier, after administered to or upon a subject, does not cause undesirable physiological effects. The carrier in the pharmaceutical composition must be “acceptable” also in the sense that it is compatible with the active ingredient and, preferably, capable of stabilizing it. One or more solubilizing agents can be utilized as pharmaceutical carriers for delivery of an active agent. Examples of other carriers include colloidal silicon oxide, magnesium stearate, cellulose, and sodium lauryl sulfate.

Pharmaceutically effective compositions of this invention may be administered to humans and other animals by a variety of methods that may include continuous or intermittent administration. Examples of methods of administration may include, but are not limited to, oral, rectal, parenteral, intracisternal, intrasternal, intravaginal, intraperitoneal, topical, transdermal, buccal, or as an oral or nasal spray. Accordingly, the pharmaceutically effective compositions may also include pharmaceutically acceptable additives, carriers or excipients. Such pharmaceutical compositions may also include the active ingredients formulated together with one or more non-toxic, pharmaceutically acceptable carriers specially formulated for oral administration in solid or liquid form, for parenteral injection or for rectal administration according to standard methods known in the art.

The term “parenteral” administration refers to modes of administration which include intravenous, intramuscular, intraperitoneal, intracisternal, intrasternal, subcutaneous and intraarticular injection and infusion. Injectable mixtures are known in the art and comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), vegetable oils (such as olive oil), injectable organic esters (such as ethyl oleate) and suitable mixtures thereof.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid and the like. It may also be desirable to include isotonic agents such as sugars, sodium chloride and the like. Prolonged absorption of the injectable pharmaceutical form may be brought about by the inclusion of agents which delay absorption such as aluminum monostearate and gelatin. Injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

In some cases, to prolong the effect of the drug, it is desirable to slow drug absorption from subcutaneous or intramuscular injection. This may be accomplished by using a liquid suspension of crystalline or amorphous material with poor water solubility. The rate of absorption of the drug then depends upon its rate of dissolution which, in turn, may depend upon crystal size and crystalline form. Alternatively, absorption of a parenterally administered drug form may be delayed by dissolving or suspending the drug in an oil vehicle.

To prepare the pharmaceutical compositions of the present invention, an effective amount of the aforementioned agent can be intimately admixed with a pharmaceutically acceptable carrier according to conventional pharmaceutical compounding techniques to produce a dose. A carrier may take a wide variety of forms depending on the form of preparation desired for administration, e.g., oral or parenteral.

Actual dosage levels of active ingredients in the pharmaceutical compositions of this invention may be varied so as to obtain amounts of the active agents which are effective to achieve the desired therapeutic response for a particular patient, compositions and mode of administration. The selected dosage level will depend upon the activity of the active agents, the route of administration, the severity of the condition being treated and the condition and prior medical history of the patient being treated. However, it is within the skill of the art to start doses of the agents at levels lower than required to achieve the desired therapeutic effect and to gradually increase the dosage until the desired effect is achieved. A greater discussion of preferred, but not limiting, dosages is provided below.

Compositions according to the present invention may also be administered in combination with other agents to enhance the biological activity of such agents. Such agents may include any one or more of the standard anti-HIV agents which are known in the art, including, but not limited to, azidothymidine (AZT), dideoxycytidine (ddC), and dideoxyinosine (ddI). Additional agents which have shown anti-HIV effects and may be combined with compositions in accordance to the invention include, for example, raltegravir, maraviroc, bestatin, human chorionic gonadotropin (hCG), levamisole, estrogen, efavirenz, etravirine, indomethacin, emtricitabine, tenofovir disoproxil fumarate, amprenavir, tipranavir, indinavir, ritonavir, darunavir, enfuvirtide, and gramicidin.

The studies reported herein, and discussed in greater detail in U.S. application Ser. Nos. 13/271,190 and 10/581,658 the contents of which are incorporated by reference herein in their entirety, support the use of activators of apoptosis for the ablation of pathogenic HIV-infected cells that destroy the immune system. Thus, deferiprone, ciclopirox or hydralazine can be administered in combination with an apoptosis inducer or activator. Examples include cytotoxic antibiotics, such as anthracycline (doxorubicin, idarubicin, and mitoxantrone), those targeting the endoplasmic reticulum (ER) (thapsigargin, tunicamycin, brefeldin), those targeting mitochondria (arsenite, betulinic acid, C2 ceramide) or those targeting DNA (Hoechst 33343, camptothecin, etoposide, mitomycin C). Additional examples include chemotherapeutic agents, antimitotic agents, DNA intercalating agents, taxane, gemcitabine, alkylating agents, platin based components such as cisplatinum and preferably oxaliplatinum and a TLR-3 ligand. Other examples include Actinomycin D, Camptothecinm, Cycloheximide, Dexamethasone, Etoposide, Staurosporine, Colchicine, Doxorubicin.HCl, Genistein, Genistein, Okadaic acid, Phorbol-12-myristate13-acetate (PMA), Anisomycin, Tamoxifen citrate, Betulinic acid, Thapsigargin, Rosiglitazone, Brefeldin A, lonomycin, Rapamycin, Tyrphostin, and Mitomycin C. See, e.g., Casares et al. J Exp Med. 202, 1691-701 (2005) and US Application NO. 20100016235.

A “subject” refers to a human and a non-human animal. Examples of a non-human animal include all vertebrates, e.g., mammals, such as non-human primates (particularly higher primates), dog, rodent (e.g., mouse or rat), guinea pig, cat, and non-mammals, such as birds, amphibians, reptiles, etc. In a preferred embodiment, the subject is a human. In another embodiment, the subject is an experimental animal or animal suitable as a disease model (such as non-human primates). A subject to be treated can be identified by standard diagnosing techniques for the disorder.

“Treating” or “treatment” refers to administration of a compound or agent to a subject, who has a disorder (such as an HIV infection), with the purpose to cure, vaccinate, alleviate, relieve, remedy, delay the onset of, prevent, or ameliorate the disorder, the symptom of the disorder, the disease state secondary to the disorder, or the predisposition toward the disorder. The terms “prevent,” “preventing,” “prevention,” “prophylactic treatment” and the like refer to reducing the probability of developing a disorder or condition in a subject, who does not have, but is at risk of or susceptible to developing a disorder or condition. A “therapeutically effective amount” refers to the amount of an agent sufficient to effect beneficial or desired results. A therapeutically effective amount can be administered in one or more administrations, applications or dosages and is not intended to be limited to a particular formulation or administration route.

The agent can be administered in vivo or ex vivo, alone or co-administered in conjunction with other drugs or therapy. As used herein, the term “co-administration” or “co-administered” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

In an in vivo approach, deferiprone, ciclopirox or hydralazine may be administered to a subject. Generally, the agent is suspended in a pharmaceutically-acceptable carrier (e.g., physiological saline) and administered orally or by intravenous infusion, or injected or implanted subcutaneously, intramuscularly, intrathecally, intraperitoneally, intrarectally, intravaginally, intranasally, intragastrically, intratracheally, or intrapulmonarily. In an ex vivo approach, a subject's blood can be withdrawn and treated with the above-mentioned agent and then the blood thus-treated is given back to the subject.

The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.01-100 mg/kg. Variations in the needed dosage are to be expected in view of the variety of agents available and the different efficiencies of various routes of administration. Variations in these dosage levels can be adjusted using standard empirical routines for optimization as is well understood in the art. Encapsulation of the agent in a suitable delivery vehicle (e.g., polymeric microparticles or implantable devices) may increase the efficiency of delivery.

In certain embodiments, the dose of deferiprone, ciclopirox or hydralazine is adjusted to achieve an in vivo serum concentration that is effective to produce an apoptotic response sufficient to stimulate the immune system against the retrovirus to levels effective to reduce the viral load to at least about 10% versus baseline and to activate the subject's natural immunological responsiveness to the virus. In another embodiment the viral load reduction is to at least about 1% versus baseline; in a further embodiment the viral load reduction is to at least about 0.1% versus baseline; in a still further embodiment the viral load reduction is to at least about 0.01% versus baseline. In one embodiment a blood serum concentration of deferiprone, ciclopirox or hydralazine of at least about 125 micromolar is achieved. In another embodiment a serum concentration of deferiprone, ciclopirox or hydralazine of at least about 150 micromolar is achieved; in a further embodiment a serum concentration of deferiprone, ciclopirox or hydralazine of at least about 175 micromolar is achieved. In another embodiment a serum concentration of deferiprone, ciclopirox or hydralazine of at least about 200 micromolar is achieved; in a further embodiment a serum concentration of deferiprone, ciclopirox or hydralazine of at least about 225 micromolar is achieved; in a still further embodiment a serum concentration of deferiprone, ciclopirox or hydralazine of at least about 250 micromolar is achieved. The oral dose to achieve this serum concentration can be from about 10 mg/kg to about 100 mg/kg or greater, preferably about 20 to about 75 mg/kg; more preferably about 30 to about 50 mg/kg. In one embodiment, the oral dose is about 33 mg/kg. In another embodiment the oral dose is about 50 mg/kg. These amounts are effective to produce an immune response effective to maintain a reduction in antiviral load of at least about 90% preferably a reduction of at least about 99%, more preferably a reduction of at least about 99.9%; most preferably a reduction of at least about 99.99% for at least about 3 months to about 1 year or longer after drug cessation and excretion. In one embodiment the immune system effect lasts at least about 3 to about 6 months; preferably about 6 to about 12 months after dosing ceases, yet may show the same individual variability as immunization with an exogenous vaccine.

Unlike microbicides and antiretroviral therapy (ART), which require constant adherence, as a prophylaxis the active agent described herein can be administered once or a few times in a short course, soon after virus exposure or during the early phases of the infection, in order to purge a substantial fraction, if not all, of virus-harboring cells from the infected individuals. A significant reduction of viral burden in HIV-infected individuals should have a significant impact in preventing or delaying disease progression of these individuals, as well as reducing virus transmission to the community. The above-described compounds may also be applied as a therapeutic agent, in conjunction with or after successful ART to eradicate most, if not all, virus-infected cells that remain. Hence, the use of therapeutic agent has the potential to complement, shorten, or perhaps eliminate, ART, which is currently considered to be lifelong. The use of therapeutic agent also has the potential to be effective when conventional ART has elicited drug- or multidrug resistance of HIV, and therefore is failing as a therapeutic life-saving option; in such setting, use of therapeutic agent is expected to ablate the HIV-infected cells harboring and producing ART-resistant virus when administered as part of a salvage regimen.

In one example, deferiprone, ciclopirox or hydralazine may be administered for at least about 1 week, a least about 2 weeks, at least about 3 weeks, at least about 4 weeks or longer, or at least about a month or longer, or any length of time necessary to reduce viral load below a desired threshold. The capacity of the agent to purge a substantial fraction of virus-harboring cells from the infected individuals has a considerable impact in delaying disease progression and decreasing the duration of ART in these individuals, as well as reducing virus transmission to the community. In certain aspects, deferiprone, ciclopirox or hydralazine is administered until the level of viral load (HIV RNA) is reduced to at least 10%, at least 20%, at least 30%, at least 40%, or at least 50% below the expression level prior to treatment. In certain aspects, p24 expression levels continue to decline further after, and despite of, administration of deferiprone, ciclopirox or hydralazine has ceased.

### EXAMPLES

### Materials and Methods

Reagents and cells. Drug-grade deferiprone was kindly provided by Apotex (Toronto, Canada). PBMCs were isolated from the blood of healthy donors, per an IRB-approved protocol, and stimulated overnight with phytohemagglutinin (PHA) and human IL-2. Stimulated cells were pelleted and resuspended for culture at a final concentration of 0.5×10⁶ cells/ml in PHA-free RPMI 1640 medium containing 10% fetal calf serum (v/v), 100 units/ml penicillin G, 100 μg/ml streptomycin, 2 mM glutamate, and 3.5 ng/ml human IL-2 (Medium B). Cultures were incubated at 37° C., 5% CO₂, and 95% humidity.

To obtain clinical isolates of HIV-1, donors were consented to provide blood samples per an IRB-approved protocol. For infection, 5×10⁶ uninfected stimulated PBMCs were co-cultured with 10×10⁶ PBMCs from one of the HIV-infected donors in Medium B. On day 3, half of the supernatant was removed and replenished with an equal volume of fresh Medium B. On day 7, the medium was likewise replenished and 7.5×10⁶ stimulated uninfected PBMCs were added. On days 10, 17, and 24, half of the supernatant was replenished. On days 14, 21, and 28, stimulated uninfected PBMCs were added. Cells were harvested when p24 reached 250 pg/ml, cryopreserved in freezing medium (90% fetal calf serum, 10% dimethyl sulfoxide), and stored in liquid nitrogen as infected PBMC stock. Cell-free supernatants were stored at −80° C.

To establish persistent infection of cultured PBMCs, uninfected cells (5×10⁶ cells) and infected PBMC stock (0.5×10⁶ cells) were co-incubated in a 25 ml culture flask at a final concentration of 0.22×10⁶ cells/ml. Cultures were allowed to establish productive infection, defined by medium p24 at or above 250 pg/ml, and deferiprone was added. Cultures were replenished with Medium B and freshly isolated uninfected PBMCs on alternate days. For replacement of Medium B, half of the supernatant was gently exchanged without disturbing the cells, and the drug concentration was adjusted appropriately. For replacement with freshly isolated, stimulated and uninfected PBMCs, half of the cells and supernatant were removed and replaced with 2.5×10⁶ cells in the proper volume of Medium B, with adjustment of the drug concentration. Cell-free supernatants were saved for p24 and viral RNA measurements.

Quantitation of cell number, viability, diameter, and apoptotic volume decrease. These parameters were measured in PBMC cultures by computerized image analysis of trypan blue exclusion (VI-CELL™; Beckman Coulter; Fullerton, Calif.).

DNA fragmentation assay. Apoptotic DNA fragmentation was quantified flow-cytometrically, using a TUNEL (terminal deoxynucleotide transferase dUTP nick end-labeling) assay (APO-BRDU™; Phoenix Flow Systems; San Diego, Calif.).

Detection of p24, viral RNA, and viral DNA. p24 core antigen in the supernatant was quantified by ELISA (Retrotek HIV-1 p24™; ZeptoMetrix Corp.; Buffalo, N.Y.). HIV-1 RNA copy number in the supernatant of PBMC cultures and in plasma of patients enrolled in the exploratory deferiprone trial was determined with a PCR-based and FDA-approved assay (Amplicor HIV-1 Monitor™; Roche Diagnostics Corp.; Indianapolis, Ind.). For clinical samples, the assay was used per the Standard Specimen Processing Procedure (sensitivity limit 400 copies/ml); for cell culture samples, the assay was used in both the Standard Specimen Processing and the UltraSensitive Specimen Processing Procedure (sensitivity limit 50 copies/ml). The Roche Amplicor HIV-1 DNA Test (Roche Diagnostics Corp.; Indianapolis, Ind.) was used for qualitative detection of HIV-1 DNA.

Measurement of cytotoxicity in epithelial cell culture. The human uterine epithelial cell line ECC-1 was cultured in transwell inserts in special, insert-accommodating 24-well plates (Fisher Scientific; Pittsburgh, Pa.) as described. This established an epithelial barrier-forming system of polarized, tight junction-linked human epithelial cells with both apical and basolateral compartments. As an indicator of tight junction formation, trans-epithelial resistance (TER) was measured using an EVOM electrode and Voltohmmeter (World Precision Instruments, Inc., Sarasota, Fla.). Once the seeded ECC-1 reached maximal epithelium-like barrier function, ascertained by TER≧1000 ohms/cm², drugs were added to some wells and TER measurements taken on consecutive days. Medium supplemented with the appropriate amount of drug was replenished every day in the apical chamber, and every other day in the basolateral chamber. At least two independent experiments were conducted with a minimum of 4 wells per drug or control.

### Example 1

In order to assess suppressive effects of deferiprone on the formation of HIV-1 RNA and HIV-1 protein, deferiprone was tested in vitro at 100 μM and 200 μM; below 100 μM p24 expression and viral copy number were marginally affected. (A deferiprone peak serum concentration of 200 μM has been observed occasionally, but this level is uncommon in thalassemic patients.) In cultures of human peripheral blood mononuclear cells (PBMCs) stably infected with clinical isolates of HIV-1, both concentrations reduced p24 to ≦10% of controls within two weeks, at which time HIV-1 RNA monitoring was begun (FIG. 1). Month-long monotherapy with deferiprone did not elicit viral breakthrough (FIG. 2), in contrast to monotherapy with suppressive antiretrovirals, e.g. nevirapine or zidovudine. In infected replenished cultures treated with 100 μM deferiprone, the viral copy number decreased by just one log below the self-sustaining set-point of controls, despite weeks of maximal p24 suppression (FIG. 2). Post-treatment rebound reacquired that set-point within 72 hours (FIG. 1, green line segment). Cessation of suppressive antiretrovirals, as monotherapy or in combination, results in similarly rapid resurgence of HIV-1 production in culture. By contrast, at 200 μM the viral copy number declined continuously, falling by three logs (FIG. 1A, lower panel). Upon reaching 10³ copies/ml, deferiprone was discontinued. Despite absence of drug and replenishment of CD4-positive lymphocytes, HIV-1 RNA did not rebound. Instead, it remained at the post-treatment level during further sampling for 11 days. The small yet consistently reproduced, continuous decline in viral RNA immediately post-treatment (FIGS. 1 and 2a) suggests a process that, once triggered by a threshold concentration X (100 μM≦X≧200 μM), continues to exert effects in the immediate post-treatment period. Deferiprone blocks viral resurgence in a concentration-dependent manner by eliminating virally infected cells. This elimination may be complete at the time of treatment cessation and/or the process (apoptosis of virally infected cells, see below) may extend beyond the period of treatment.

It was then determined whether the persistent suppression after cessation of 200 μM deferiprone was due to elimination of HIV-1 DNA, the genetic prerequisite for viral rebound. HIV-1 DNA detected by standard nucleic amplification assays sensitively detected both unintegrated and integrated viral genes that have been reversed transcribed following viral entry into cells. In repeated experiments to assess the effect of 200 μM deferiprone on HIV-1 DNA, viral RNA copies in infected untreated controls were detected in the 106/ml range whereas in infected 16-day treated cultures, they were reduced by three orders of magnitude. HIV-1 DNA in post-treatment cultures was either negative or at the lower limit of detectability, but was strongly positive in untreated controls during 10 days of post-cessation monitoring (FIG. 1A). Monotherapy with 200 μM deferiprone markedly reduces or eliminates HIV-1 DNA in primary cell cultures previously productively infected with clinical isolate. A similar depletion of HIV-1 infectivity in culture requires the combination, or the alternating use, of several suppressive antiretrovirals so as to forestall the selection of drug-resistant escape mutants.

To determine whether viral RNA suppression by 200 μM deferiprone, upon reaching a nadir of 10³ copies/ml and continued observed decline to <10² copies/ml (FIGS. 1 and 2A), resulted in reduced or terminated infectivity, post-treatment replenishment was prolonged from 10 days to 3 months, guided by clinically observed viral rebound delays of up to 50 days. Depending on the particular isolate—primary cell combination, the nadir of <10³ copies/ml was reached after 2-4 weeks. During variably extended off-drug periods, re-emergence of HIV-1 RNA was monitored in the replenishment model (data not shown). However, even after 90 days off-treatment, no re-emergence of productive infection in the previously productively infected cultures was observed, despite persistence of the PCR signal at the limits of detection in assays of different sensitivity (FIG. 2B).

The depletion of the HIV-1 DNA signal suggests a decline or elimination of infected cells and its irreversibility is compatible with their depletion by cell death. However, computerized trypan blue-based cell analysis showed viability in these cultures to remain invariably at or above 95%, and apoptotic volume decrease was not detectable (data not shown). These negative findings are attributed to the protocol-required replenishment with freshly isolated primary cells. Therefore non-replenished short-term primary cell cultures, uninfected or infected in vitro with clinical isolate, were assayed for evidence of apoptosis induced by 200 μM deferiprone. Consistent with the findings of preferential mitochondrial activation of apoptosis and its execution in HIV-infected T cell lines, the deferiprone-induced apoptosis in infected PBMCs significantly (P≦0.009) exceeded that in uninfected PBMCs. To establish apoptotic death of non-hematologic human cells triggered by deferiprone, its effect on the resistive integrity of the barrier formed in a dual-chamber system by the single layer of human epithelial cells, a verified model for drug toxicity to human tissue was studied. Exposure to 200 μM deferiprone for six days did not reduce transepithelial resistance beyond the spontaneous decay observed in untreated controls or those exposed to deferoxamine, a medicinal iron chelator like deferiprone (data not shown). Deferiprone at 200 μM does not induce apoptosis indiscriminately. Although not wishing to be bound by any theory, it is believed that deferiprone eliminates the proviral reservoir by preferential apoptotic ablation of infected lymphocytes, thereby precluding post-treatment rebound.

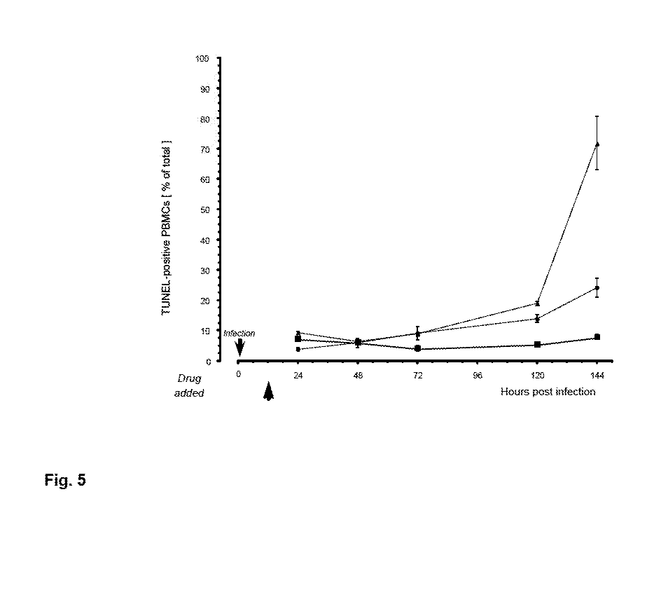

Further, at least two drugs, deferiprone and ciclopirox, both of which are hydroxy-pyridinones, induce apoptosis preferentially of freshly obtained human primary target cells (peripheral blood mononuclear cells, PBMCs) acutely infected with patient-derived HIV-1 isolates. This is shown in the FIG. 5 for the case of 30 μM ciclopirox. Less than 10% of cells in infected untreated cultures (black squares) are apoptotic at the height of retroviral multiplication, 144 hours after inoculation. By contrast, at that time 71.8±8.8% of all cells in infected treated cultures (black triangles) had fragmented their DNA, i.e. become apoptotic, compared with 24.1±3.2% in uninfected treated cultures (black circles) [P=0.009; FIG. 5]. Thus, it was the presence of HIV-1 in PBMC cultures that markedly increased the susceptibility to ciclopirox-triggered apoptosis. Similar results were obtained for deferiprone at a concentration in the range of 200 μM. These findings confirm the precedent that specific compounds can effect blockade of, and promote cells' escape from, retroviral anti-apoptosis.

Use of deferiprone or cyclopirox without interruption for up to three weeks reduces the number of infective virions produced to undetectable levels. These remain unchanged even after drug discontinuation for at least three months (data not shown). In a pilot experiment, we attempted to re-infect these HIV-1 experienced cell cultures after drug discontinuation, when endogenous infectious virus was at the limit of detectability. The susceptibility to reinfection, as assessed by p24 levels, was markedly reduced, and after one week p24 levels in reinfected, previously HIV-1 experienced cultures were less than 30% of those in controls. Despite the significant limitations of this pilot experiment, such as its mixed lymphocyte culture design and the obvious dearth of antigen presenting cells in this system, the results were encouraging enough to motivate further, more precisely targeted experimentation.

### Example 2

### A. Exploratory Trial of Deferiprone

The Galenic preparation used was the oral formulation of deferiprone marketed by Apotex (Toronto, Canada) as immediate-release 500 mg tablets (Ferriprox™). The pilot trial was designed as a single-center, double-blind, placebo-controlled, two-stage study, investigating the safety, tolerability, antiretroviral activity, and pharmacokinetic profile of deferiprone in asymptomatic HIV-infected antiretroviral-naive persons.

A total of 26 healthy and HIV-infected consenting volunteers were studied in a specialized unit for Phase I drug trials. Two oral dose levels of deferiprone were tested, 33 mg/kg (33 mg/kg QD on days 1 and 7, and 33 mg/kg TID on days 2-6 [seven asymptomatic HIV-infected antiretroviral-naive persons]) and 50 mg/kg (50 mg/kg QD on days 1 and 7, and 50 mg/kg TID on days 2-6 [six healthy volunteers and seven asymptomatic HIV-infected antiretroviral-naive persons, four of whom completed the treatment period]). The placebo controls comprised a total of six persons (two healthy volunteers and four asymptomatic HIV-infected antiretroviral-naive persons, three of whom completed the treatment period).

The protocol specified an interim safety evaluation of all subjects on 33 mg/kg by a safety committee (see Supporting Information, Supplementary Text 3). Only in the absence of safety concerns at the 33 mg/kg dose level did enrollment begin at the 50 mg/kg dose level. Safety evaluations did not involve a formal statistical analysis and were disclosed only to relevant individuals in order to make decisions regarding safety. Investigators and study personnel were not privy to any unblinded data. Each cohort was unblinded only at study completion. Enrollment was limited to age 18 to 60 years with a minimum of 12 evaluable deferiprone-treated individuals. The protocol involved a screening visit; a pharmacokinetic study requiring confinement for 12 hours with multiple blood draws from a peripheral vein after oral intake of the first deferiprone dose, either 33 mg/kg or 50 mg/kg; an on-drug treatment period of one week (first stage of protocol), which comprised at least three repeat visits; an off-drug observation period of seven weeks (second stage of protocol), which comprises at least two repeat visits; and an exit visit. Primary parameters included safety and tolerability (e.g. vital signs, laboratory variables, cardiac monitoring by ECG) and antiretroviral activity (e.g. RNA copies of HIV). Secondary parameters included pharmacokinetic variables (e.g. peak serum concentration) in serum, drawn after initial drug ingestion at fixed hours (0, 0.5, 1, 1.5, 2, 3, 4, 6, 9, 12). The concentration of the nonconjugated, chelation-competent form of deferiprone in serum was determined by UV detection at 280 nm, after separation via reversed-phase HPLC under isocratic conditions. The lower limit of deferiprone quantitation was 1.466 μM, or 0.204 μg/ml. Viral load was measured as described above.

### B. Data Analysis

For the cell culture experiments with deferiprone and the exploratory clinical trial, descriptive statistics were generated using Microsoft Excel 2011. In the exploratory trial, outcome analysis was performed to assess the intercohort and intraindividual virological response to deferiprone, using Student's t-test for log₁₀-based analysis of the time-specified differentials (Δ log) between the cohort identified medians (±standard error of the mean [SEM]) generated from the multiple measurements per subject on treatment minus before treatment. (‘response’ in the first stage of the protocol) and off treatment minus before treatment (‘rebound’ in the second stage of the treatment). P<0.05 was used as criterion for statistical significance. To assess the in vivo antiretroviral activity of deferiprone in a hypothesis-based manner, the number of subjects was analyzed by two-sided Fisher's Exact Test who did or did not achieve a postulated antiretroviral threshold concentration x, with x≧150 μM as cut-off. To assess the in vivo antiretroviral activity of deferiprone in a hypothesis-independent manner, the Δ log of the HIV-1 RNA levels achieved in the first and the second stage of the protocol was analyzed after post-treatment segregation into ‘responders’ and ‘non-responders’ per the discontinuation trial design (DTD).

### C. Ethics Statement

The human protocol for the primary cell culture experiments was conducted at the University of Medicine and Dentistry of New Jersey, New Jersey Medical School. Newark, N.J., and covered the isolation and handling of mononuclear cells from peripheral blood of HIV-1 infected and uninfected volunteers. Blood draws from an antecubital vein for the purposes of this study were approved by the university's IRB (#0119990009).The human protocol for the exploratory trial of deferiprone was approved by the Ethics Committee of the Faculty of Health Sciences, University of the Free State, Bloemfontein, South Africa (Protocol #LA-26-106/83107) and passed review by the Institutional Review Board of the University of Medicine and Dentistry, Newark, N.J., United States of America (Protocol #Pro2012002121). The protocol was implemented by the research contract organization Parexel International (Lowell, Mass.) as Study No. 83207 in compliance with the Declaration of Helsinki as set forth by the statutory requirements of the governmental Health Professions Council of South Africa (HPCSA), which legally guide the process of obtaining informed consent from research subjects, in particular in sections 3-7 and 12-18. Written informed consent was obtained from each person before enrollment.

### D. Results and Discussion

The existence of an antiretrovirally effective threshold concentration between 100 μM and 200 μM (FIG. 1) and, upon drug cessation, the lack of rebound to the pre-treatment set-point of viral RNA copies (FIG. 2A,B) informed the design of a double-blind placebo-controlled pilot human trial. An in vivo threshold at ≧150 μM for antiretroviral activity was hypothesized since in infected T-cell lines, effective suppression of HIV-1 generation and selective induction of apoptosis both require at least 150 μM deferiprone. The minimal antiretroviral activity of deferiprone monotherapy for a week should, only in individuals with serum concentrations above this threshold, cause a viral load decline at least in the range of zidovudine monotherapy, i.e. −0.3 log₁₀, an apparently minor change that nevertheless reduces the annual risk of progression to AIDS-related death by 25%.

In the IRB-approved pilot trial, HIV-1-infected but treatment-naïve individuals who achieved ≧150 μM in serum (Group A) on either 33 mg/kg po TID or 50 mg/kg po TID oral deferiprone (N=7), demonstrated a modest virological effect during the seven-day [D] treatment [Δ log₁₀ D7 vs. D1: −0.40±0.1], and 6 of 7 experienced an acute viral load decline [Δ log 10 D7 vs. D1: −0.42±0.05]. By contrast, the cohort that did not achieve ≧150 μM in serum (Group B), receiving either placebo (N=3) or deferiprone (N=4), did not show an acute virological effect [Δ log 10 D7 vs. D1: −0.07±0.05 and +0.17±0.07, respectively] (FIG. 3). Individuals in the 50 mg/kg po TID group did not tolerate the regimen, resulting in protocol disruptions and early terminations, yet invariably only deferiprone peak concentrations ≧150 μM coincided with an acute decline in HIV-1 RNA (Δ log 10=−0.44±0.1). The viral response distribution relative to the serum deferiprone levels among the 11 treated subjects is significant (P=0.0152) by two-sided Fisher's Exact test and thus, consistent with the hypothesis of an antiretrovirally effective in vivo threshold at ≧150 μM deferiprone.

To analyze the pilot trial in a hypothesis-independent manner, the two-stage discontinuation trial design (DTD) was followed. In a first stage of predetermined duration, all subjects receive the new agent; in the second stage, subjects are segregated into ‘responders’ and ‘non-responders’ and their subsequent outcomes are followed separately in order to avoid dilution of the primary data for treatment benefit, if it exists. This enrichment approach is descriptive and ignorant of mechanistic knowledge. The results of the DTD-based analysis for deferiprone are shown in FIG. 4. At the end of the first stage, the 6 responders and the 5 non-responders differed by Δ log 10=0.64 (D7 vs. D1, P=0.00007). At the end of the second stage, the 5 responders—one was lost (Subject 2)—and the 5 non-responders differed by Δ log=0.54 (D53 vs. D1, P=0.008) even though one responder (Subject 22) had reacquired the pre-treatment level.

In summary, 80% of acute responders to the brief, one-week deferiprone pulse did not reacquire their viral load baseline for seven weeks post-treatment, i.e. at 200 times the half-life of said medicine in the human body and thus assuredly after its total excretion, indicating the acquisition of autonomous endogenous suppression of the HIV-1 viral load, and thereby producing “vaccineless vaccination”. The acquisition of autonomous endogenous suppression, as in any vaccination, is further evidenced by the fact that the viral load is not just blocked from rebounding, but can decline further, entirely off drug and “on its own”, as evidence by subjects #1 and #24 (FIG. 4).

Although not wishing to be bound by any particular theory, in order to explain this unanticipated discovery—after discontinuation of ART, HIV RNA and HIV protein (e.g., p24) resurge rapidly, making cure principally impossible—, the following mechanistic considerations are advanced. Deferiprone has now been shown to inhibit the expression of the HIV genome and to cause the ablation of HIV-infected cells by apoptosis. This may be construed to indicate immunogenic activity in vivo as follows: When introduced into a host with a functioning immune system, HIV-infected PBMCs rendered apoptotic ex vivo induce HIV-1 specific cellular and humoral responses that effectively protect against challenge with live HIV-infected cells. Similarly, infected cells rendered apoptotic in vivo by compounds such as deferiprone and ciclopirox might serve as vehicles that deliver retroviral immunogens to an immune system that has, at least temporarily, been de-paralyzed by the same drugs via their ability to inhibit HIV-1 gene expression.

Such an explanation is insufficient, however, since HIV-1 virions and apoptotic cells cover themselves with a molecule that cloaks them from being recognized as immunogens by the immune system. This molecule, Clq, is avidly bound by the gp41 element of HIV-1 as well as by human cells undergoing apoptosis. Cells undergoing apoptosis are a most common event in the human body and, as long as these dying cells are covered by Clq, they do not elicit immune system activation against self-antigens. However, hereditary defects of Clq expression in humans lead invariably to autoimmune disease; in fact, such deficiencies make human Clq one of the strongest disease susceptibility genes for autoimmune disease. With Clq cloaking being in effect, the explanation presented above would therefore be invalid.

However, it is now believed that deferiprone inhibits the key event in Clq biosynthesis, the posttranslational hydroxylation of specific prolyl residues in its collagen-like domains by the enzyme prolyl hydroxylase. Of note, DEF-treated thalassemic patients display signs of immune activation and launch immune responses against self-epitopes. The mechanistic concept for vaccineless vaccination against HIV-1 therefore comprises at least three elements: (i) activation of apoptosis preferentially in HIV-infected cells; (ii) inhibition of HIV-1 gene expression and therefore, temporary relief from its immunosuppressive products; and (iii) limitation of the HIV-1 protecting self-tolerance via suppression of Clq biosynthesis.

What is claimed is:

1. A method of inducing an anti-retroviral immune response by counter-acting retro-virus induced anti-apoptosis comprising:
administering a therapeutic agent selected from the group consisting of deferiprone, ciclopirox, hydralazine and combinations thereof to a subject infected with HIV-1 in an effective amount and for a time period effective to allow infected cells to present HIV-1 antigens for immunological stimulation, followed by discontinuing administration of said deferiprone, ciclopirox or hydralazine after said effective time period, whereby viral load decreases during the administration and continues to decrease after the deferiprone is excreted from the subject's body.

2. The method of claim 1, wherein the concentration of deferiprone, ciclopirox or hydralazine in a serum of the subject is at least about 150 μM.

3. The method of claim 2, wherein the concentration of at least about 150 μM is substantially maintained within the subject for a period of at least about one week.

4. The method of claim 2, wherein the concentration of at least about 150 μM is substantially maintained within the subject until decline occurs in expression of the human genome-integrated HIV DNA, as monitored by p24 or HIV RNA levels, or a reduction in that viral DNA itself.

5. The method of claim 1, wherein the concentration of deferiprone, ciclopirox or hydralazine in a serum of the subject is at least about 200 μM.

6. The method of claim 5, wherein the concentration of at least about 200 μM is substantially maintained within the subject for a period of at least one week.

7. The method of claim 5, wherein the concentration of at least about 200 μM is substantially maintained within the subject until decline occurs in expression of the human genome-integrated HIV DNA, as monitored by p24 or HIV RNA levels, or a reduction in that viral DNA itself.

8. The method of claim 1, wherein the dosage administered to the subject is from about 10 mg/kg bodyweight per day to about 50 mg/kg bodyweight per day.

9. The method of claim 1, wherein the dosage administered to the subject is from about 40 mg/ml to about 140 mg/kg bodyweight per day.

10. The method of claim 1, wherein the time period for administration is at least about a week.

11. A method of inducing an anti-viral immune response by limiting self-tolerance protection of viruses comprising:
administering deferiprone, ciclopirox, hydralazine or a combination thereof to a subject infected with HIV-1 in an effective amount and for a time period effective to allow infected cells to present HIV-1 antigens for immunological stimulation, followed by discontinuing administration of said deferiprone, ciclopirox or hydralazine after said effective time period, whereby viral load decreases during the administration and continues to decrease after the deferiprone is excreted from the subject's body.

12. The method of claim 11, wherein the concentration of deferiprone, ciclopirox or hydralazine in a serum of the subject is at least about 150 μM.

13. The method of claim 12, wherein the concentration of at least about 150 μM is substantially maintained within the subject for a period of at least one week.

14. The method of claim 12, wherein the peak concentration of at least about 150 μM is substantially maintained within the subject until decline occurs in expression of the human genome-integrated HIV DNA, as monitored by p24 or HIV RNA levels, or a reduction in that viral DNA itself.

15. The method of claim 11, wherein the concentration of deferiprone, ciclopirox or hydralazine in a serum of the subject is at least about 200 μM.

16. The method of claim 15, wherein the peak concentration of at least about 200 μM is substantially maintained within the subject for a period of at least about one week.

17. The method of claim 15, wherein the peak concentration of at least about 200 μM is substantially maintained within the subject until decline occurs in expression of the human genome-integrated HIV DNA, as monitored by p24 or HIV RNA levels, or a reduction in that viral DNA itself.

18. The method of claim 11, wherein the dosage administered to the subject is from about 10 mg/kg bodyweight per day to about 50 mg/kg bodyweight per day.

19. The method of claim 11, wherein the dosage administered to the subject is from about about 40 mg/ml to about 140 mg/kg bodyweight per day.

20. The method of claim 11, wherein the time period for administration is at least about a week.

21. A method of inducing an anti-retroviral immune response by counter-acting retro-virus induced anti-apoptosis comprising:
administering deferiprone, ciclopirox, hydralazine or a combination thereof to a subject infected with HIV-1 in an effective amount and for a time period effective to (i) activate of apoptosis preferentially in HIV-infected cells; (ii) inhibit HIV-1 gene expression and therefore, temporary relief from its immunosuppressive products; and (iii) limit the HIV-1 protecting self-tolerance via suppression of Clq biosynthesis; and
discontinuing administration of said deferiprone, ciclopirox or hydralazine after said effective time period, whereby viral load decreases during the administration and continues to decrease after the deferiprone is excreted from the subject's body.
